(19) United States
(12) Patent Application Publication
(10) Pub. No.: US 2007/0249979 A1
(43) Pub. Date: Oct. 25, 2007

(54) External Wrist Fixator

(76) Inventors: Javier Ara Pinilla, Santa Cruz De Tenerife (ES); Guillermo De La Barreda Lopez, La Orotava (ES); Donato Monopoli Forle, Romdu Y Cajal (IT)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/580,731

(22) PCT Filed: Nov. 25, 2003
(86) PCT No.: PCT/ES03/00598

§ 371 Date: Apr. 10, 2007

(51) Int. Cl. A61F 5/058

(52) U.S. Cl. 602/21

(57) ABSTRACT

It consists of a main articulator embodied in an extended body (1) with two identical pitches (2) whereon two spherical bearings (10) are positioned which are traversed by two bars (13) which, in turn, pass through spherical head screws (14), with these being connected to the supports (15) through respective bearings (16). A tightening screw (8) transversely mounted on the articulator (1) makes it possible to tighten the spherical bearings (10) and thus immobilise the bars (13), which, on the other end, are immobilised by the set formed by the spherical head screw (14), the bearing (16) and the support (15) itself; the latter has holes (22) through which respective nails intended for insertion into the bone pass, with these holes being oversized with respect to the nails in order to act as guides during insertion of the latter, but having the capability to be pinched during tightening of the corresponding nuts (21) for solid fixation to the nails, parallel to fixation to the corresponding bar's (13) support (15).

### PURPOSE OF THE INVENTION

The present invention relates to a device which has been especially conceived to create a rigidifying bridge between the patient's arm and hand, in order to immobilise his/her wrist, in the event of fracture or dislocation of said wrist, for the entire recovery period during which the joint must be immobilised.

Nevertheless, the device is equally applicable to a single bone, such as for instance the ulna or the radius, when there is this type of fracture.

The purpose of the invention is to allow for easy positional regulation of the two ends of the fixator in order that, once the latter is implanted on the patient's arm and hand, the pertinent regulations may be conducted for the relative position between these elements at the wrist level to be the most suitable.

### BACKGROUND OF THE INVENTION

There are known fixators with said purpose, which are also placed on the outer side of the arm and are based on the use of two pairs of nails or screws intended to be fixated to two bones or two parts of a bone, emerging on the outer side of the limb, where each pair of screws or nails receives a fixation block from which a bar transversely emerges; this bar is crowned, in turn, by another block which is substantially distant from the first one, such that the two end blocks are interconnected by means of a third bar, which is perpendicular to the preceding ones and may be fixated by means of stud bolts, thus achieving an essentially rigid structure which stabilises the two elements to be joined.

This solution, which is perfectly valid from the theoretical point of view, in practice leads to problems due to the pronounced distance between the nails that are inserted into the bone and the longitudinal bar that connects the two pairs of nails, which reduces the stability of the fixation.

On the other hand, even though this device fulfils its function in a basically satisfactory manner when it is intended to immobilise the two portions of a fractured bone, such as for instance the ulna or the radius, it is very difficult to apply, and requires great professional expertise, when the purpose is to immobilise the hand to the arm, due to a wrist problem, since, whereas in the first case it is only necessary for the two bone sectors to be aligned, in the second case, depending on the various types of injuries, the hand must adopt different inclinations or orientations with respect to the forearm.

### DESCRIPTION OF THE INVENTION

The external fixator proposed by the invention resolves in a fully satisfactory manner said problems, since it allows for a perfect positional regulation between the two anatomical elements to be joined, after insertion of the nails into them, and not only with respect to closeness/distance, but also with regard to their relative orientation.

More specifically, in order to achieve this, the fixator is structured on the basis of two supports provided with through-holes for respective pairs of the nails used for insertion into the bone; each of these supports is aided by a bearing and a screw-nut set wherein said screw incorporates a head which approaches a spherical annular configuration in order to allow for the passage of a bar which connects said support with a main articulator.

Said through-holes for the nails are laterally open, by means of a narrow slot which, upon closure, causes the support to be fixated to the nails when the screw-nut set is tightened; during this tightening the screw's spherical head substantially fits into the intermediate bearing, against which the bar laterally presses, thus becoming immobilised.

The main articulator is embodied in an extended body with two identical pitches having a concave internal surface, which are connected by means of a longitudinal slot that opens towards both of the articulator's faces. Two spherical bearings are placed on said pitches; the bars also pass through the bearings, being pressed and immobilised by means of a tightening screw which is transversely mounted on the articulator; the bars' positioning and, consequently, the distance between the supports may be regulated by acting on said tightening screw.

The two spherical bearings have a diametral slot which makes it possible to tighten and/or loosen the bars for their regulation.

As becomes evident, according to the described structuring, the distance between the two end supports may be regulated at will depending on the position of the bars that are fixated to the main articulator, upon tightening thereof, while these bars may form any angle, since they tilt on the main articulator when the latter is in a loose situation and maintain the selected angular position in a perfectly stable manner when it is definitively tightened.

The fixator's special structuring and configuration not only ensure greater mechanical stability, but also make it possible to use plastic materials to obtain certain parts thereof, with the cost reduction this entails.

### DESCRIPTION OF THE DRAWINGS

In order to complement the description that is being made, and in order to contribute to a better understanding of the invention's characteristics, in accordance with an example of preferred embodiment thereof, as integral part of this description a set of drawings is attached, wherein, for illustration purposes, but not limited thereto, the following has been represented:

FIG. 1.—Shows a perspective breakdown of the parts of an external wrist fixator embodied in accordance with the purpose of this invention.

FIG. 2.—Shows an elevation view of the main articulator which participates in said device.

FIG. 3.—Shows a section detail of the same main articulator.

FIG. 4.—Shows a cross-section detail of one of the spherical bearings which also participate in the fixator

FIG. 5.—Shows a section detail of one of the spherical annular head screws.

FIG. 6.—Shows a section detail of one of the bearings which intervenes in each of the device's support sets.

FIG. 7.—Shows a cross-section detail of one of the fixation supports for the nails.

FIG. 8.—Shows, finally, an example of practical use of the fixator, coupled to a patient's forearm and hand, according to a perspective vision.

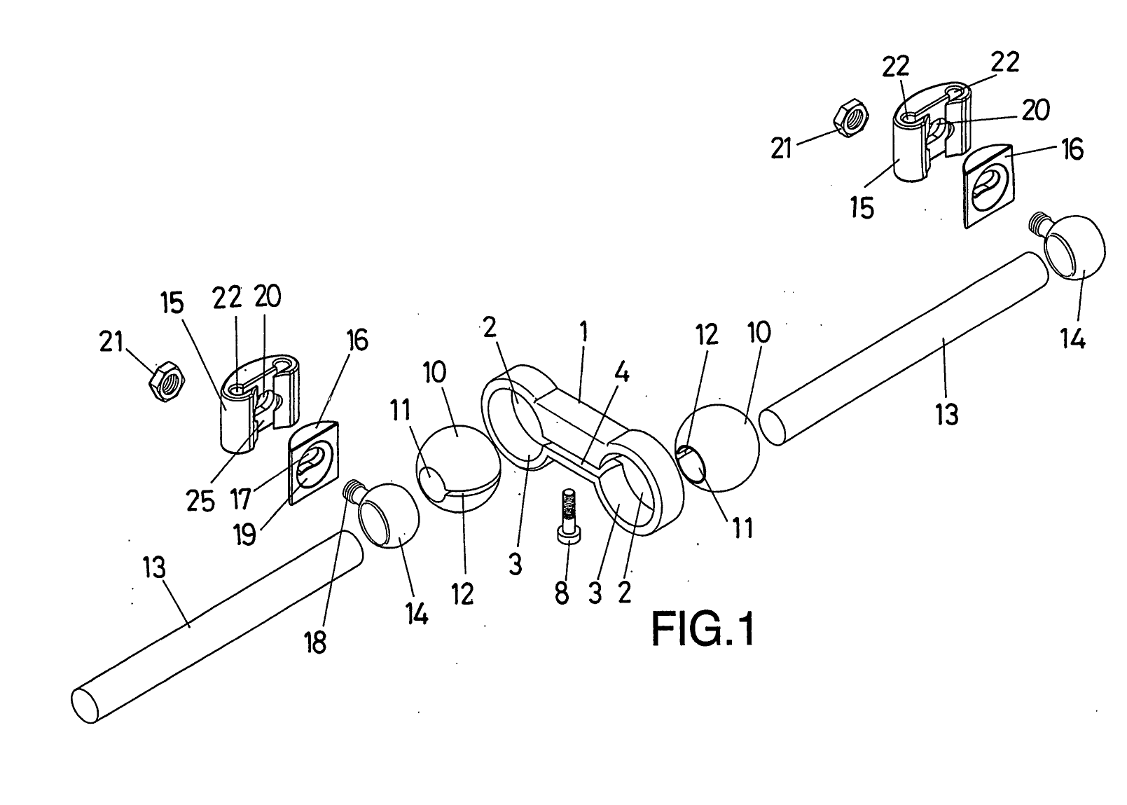

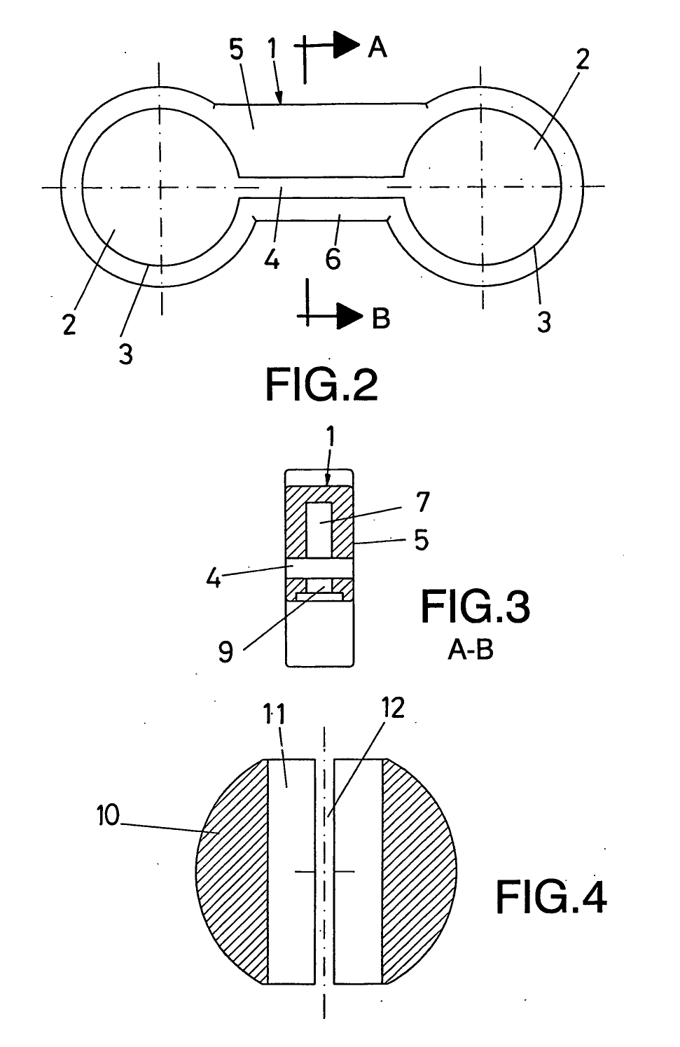

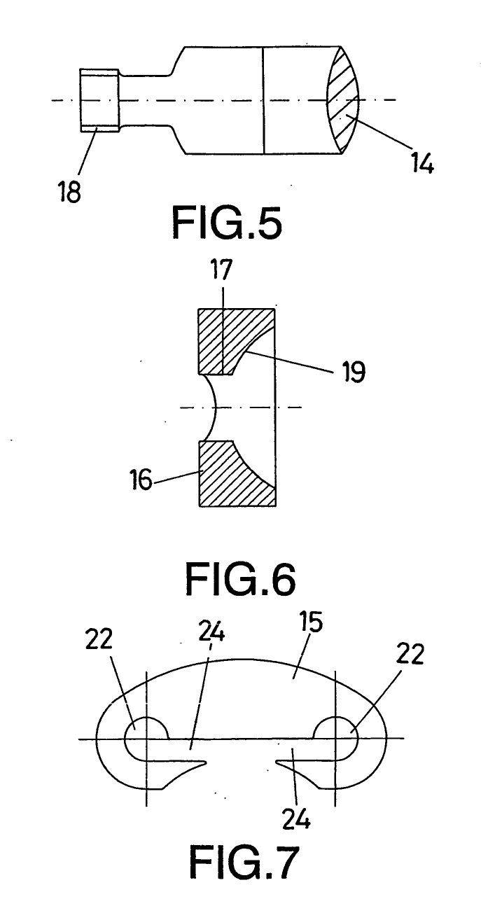

### PREFERRED EMBODIMENT OF THE INVENTION

In light of said figures, one can observe that the external wrist fixator proposed by the invention is formed by a main articulator (1), embodied in an extended body, which has a pair of identical pitches (2) with a concave internal surface (3), creating a cylindrical segment in each case. Both pitches (2) are connected by a longitudinal slot (4) that opens towards both of the articulator's (1) sides and is located close to one of the articulator's faces, creating two walls (5) and (6) thereon, the first being thicker and having a threaded, blind hole (7) for a tightening screw (8) that passes through a hole (9) on the thinner wall (6).

Two spherical bearings (10) are placed inside the spherical-segment shaped pitches (2); the bearings have a diametral through-hole (11) and a slot (12) that diametrically connects each hole (11) to the exterior, such that these spherical bearings (10) may be tightened to a greater or lesser extent by means of the screw (8); this tightening is achieved thanks to the slot (12), since tightening of the screw (8) leads to the articulator's (1) walls (5) and (6) coming closer and, thus, the pitches (2) being reduced and, consequently, the spherical bearings (10) being tightened.

One of the bars' (13) ends is housed in the spherical bearings' (10) holes (11); in turn, the bars pass through the main, spherical screws (14), all this in such a way that these bars may be introduced or removed to a greater or lesser extent by simply loosening the tightening screw (8) and performing any of those operations, in order to subsequently fixate it in an undetachable manner by means of the corresponding tightening, as previously mentioned.

In turn, the bars (13) are connected to respective supports (15), which are associated with corresponding bearings (16) provided with a slotted hole (17) in order to allow for tilting, in addition to passage of the threaded shanks (18) pertaining to the spherical screws (14) through them, in addition to the pitch or slotted hole (17), the bearings (16) have a sinkage (19) in the form of a spherical cap, which is intended to partially receive within it the screws' (14) spherical annular head.

The spherical screws' (14) threaded shanks (18), which pass through the bearings' (16) slotted holes (17), in turn pass through another slotted hole (20) provided on each of the supports (15), with tightening being performed by means of the corresponding nut (21).

The supports (15) have the special feature that the parallel holes (22) intended for passage of the respective nails (23) used for insertion into the bone are laterally open by means of respective slots (24), such that their initial diameter is slightly greater than the nails' (23), in order to act as guides upon insertion of the latter into the bone and, following the fixator's definitive positioning, tightening of the nuts (21) and, more specifically, leaning of the bearings (16) against the supports' (15) concave-curved front (25) when the slots (24) are closed, pinching of the holes (22) against the nails (23), and, consequently, relative immobilisation between these elements.

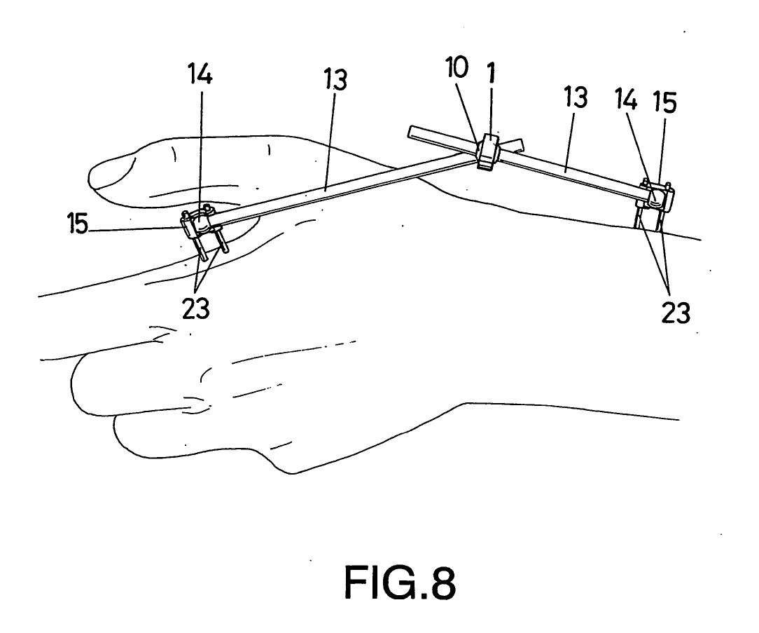

As has been previously mentioned, and as can be deduced from observing FIG. 8, the fixator makes it possible for the bars (13) to adopt any necessary angulation to be transmitted to the wrist after having inserted the nails (23) into the bone, but it may occur that this angular regulation between bars is not necessary, such as for instance in the case of a bone fracture at the forearm level, in which case it is possible to use a single bar; logically, this makes it unnecessary to use the main articulator (1), in which case the ends of said bar are simply mounted on respective sets of spherical screws (14), bearing (16), and support (15).

1. External wrist fixator which, while being especially conceived to act as a bridging element between the forearm and the hand that immobilises the wrist in any selected position, is characterised in that it is formed by a main articulator (1), whereto a pair of bars (13) are fixated by one end of both bars (13), which are capable of being regulated both longitudinally and angularly, each of both bars (13) being connected, on the other end, to a support (15) capable of being longitudinally and angularly regulated, and each of said supports (15) being provided with a pair of holes (22) for passage of the corresponding nails (23) intended for insertion into the bone; and in that the main articulator (1) is embodied in an extended body provided with a pair of identical pitches (2), whose internal surface is concave, creating cylindrical segments, connected to one another by means of an axial slot (4) which may be narrowed by tightening a transverse screw (8) in order to reduce the corresponding pitches (2), where respective spherical bearings (10) are housed with a diametral pitch (11) that is connected, through a slot (12), with the exterior in order to allow for passage and tightening of the corresponding bar (13), with the latter, in turn, passing through the pitch or hole which the head of a spherical screw (14) has for this purpose, and through which it is linked to the respective support (15).

2. External wrist fixator, as claimed in the preceding claim, characterised in that the spherical bearings (10) are located on the pitches (2) with the capacity to rotate around themselves, allowing for the bars' (13) position to be changed in any direction.

3. External wrist fixator, as claimed in claim 1, characterised in that the supports' (15) holes (22) are connected to the exterior by means of respective lateral slots (24) which, by allowing a slight lateral oversizing of said holes (22) with respect to the nails (23) intended for insertion into the bone, are pinched over said nails when they are definitively fixated to the ends of the corresponding bars (13).

4. External wrist fixator, as claimed in the preceding claims, characterised in that the supports' (15) slots (24) open towards a concave curved face (25) on which a bearing (16) traversed by the spherical annular head screw's (14) threaded shank (18) is adapted; these screws (14) are complemented by respective nuts (21).

5. External wrist fixator adapted to act as a bridging element between the forearm and the hand that immobilises the wrist in any selected position,
said fixator having a main actuator, and a pair of bars, each coupled at one end to said main actuator and having a support at the other end adapted to support a device operable to be inserted into a bone, said bars being adjustable both longitudinally and angularly in said main articulator,
each support having a spherical screw; each support capable of being longitudinally and angularly adjusted relative to its associated arm, and each of said supports being provided with a pair of holes for receiving an associated fastening device intended for insertion into a bone; and
said main articulator comprising an extended body provided with a pair of identical saddles and a transverse screw, said saddles having concave internal surfaces creating cylindrical segments, connected to one another by means of an axial slot adapted to be narrowed by tightening said transverse screw in order to close said cylindrical segments, and spherical bearings housed between said segments, with a diametral bore that is connected, through a slot with the exterior of the bearing, one of said bars being received in said bore in order to allow for tightening of said one end of the bar,
each said spherical screw having a spherical head with a cylindrical bore adapted to receive the other end of the one bar through which it is linked to the respective support.
